(19) United States
(12) Patent Application Publication
(10) Pub. No.: US 2021/0403613 A1
(43) Pub. Date: Dec. 30, 2021

(54) ONE-POT PHOTOCHEMICAL SYNTHESIS METHODOLOGY FOR CONDUCTIVE HYDROGEL FABRICATION

(71) Applicant: University of Washington, Seattle, WA (US)

(72) Inventors: Buddy D. Ratner, Seattle, WA (US); Lars Crawford, Seattle, WA (US)

(73) Assignee: University of Washington, Seattle, WA (US)

(21) Appl. No.: 17/357,887

(22) Filed: Jun. 24, 2021

Related U.S. Application Data
(60) Provisional application No. 63/044,584, filed on Jun. 26, 2020.

(51) Int. Cl. C08F 2/44; C08G 61/12; C08F 20/06; C08F 12/30; C08K 5/00; C08L 33/10; C08F 2/50

(52) U.S. Cl. CPC C08F 2/44; C08G 61/126; C08F 20/06; A61L 31/145; C08K 5/0025; C08L 33/10; C08F 2/50; C08F 12/30

(57) ABSTRACT

Methods for making a conductive hydrogel, comprising photochemically polymerizing polymerizable hydrogel-forming agents in the presence of a polymerizable conductive monomer to provide a conductive hydrogel; liquid resins for preparing conductive hydrogels comprising photochemically polymerizable hydrogel-forming agents and polymerizable conductive monomers; conductive hydrogels prepared by the methods or from the liquid resins; and electrodes comprising the conductive hydrogels.

### CROSS-REFERENCE TO RELATED APPLICATION

This application claims the benefit of U.S. Application No. 63/044,584, filed Jun. 26, 2020, expressly incorporated herein by reference in its entirety.

### BACKGROUND OF THE INVENTION

Tens of millions of people worldwide suffer from neurological diseases and injuries such as spinal cord injury, stroke, Parkinson's, epilepsy, and multiple sclerosis among others. Brain-computer interfaces (BCIs) provide an opportunity to bypass faulty neuronal circuitry to return bodily function or interact with and/or control external devices. BCIs are designed to record neural signals and decode information content to drive a computer cursor, a robot arm, electrical stimulation of muscles, or another actuator.

To date, penetrating microelectrode arrays (MEA) which utilize the shank-like geometry provide the highest degree of specificity in recording and stimulation of individual neurons and associated neural circuitry. However, penetration into the brain parenchyma explicitly induces tissue damage which results in an immune response that may range from moderate to severe depending on the degree of implant-induced tissue damage and material properties of the electrode. For example, classical penetrating MEAs have typically been fabricated from silicon and/or metallic materials that present a major mechanical mismatch with neural tissue. This has the tendency to cause continued tissue damage beyond the initial implantation as a result of normal brain micromotions during respiration and metabolism. Both the initial implantation and this prolonged damage have the propensity to induce a chronic form of neuroinflammation, known as gliosis.

In the context of neural electrode implantation and recording, gliosis is an adverse phenomenon with a dynamic time-course of events and is known to be a primary failure mode for intracortical MEAs. Gliosis results in the formation of a cellular sheath, known as a glial scar, which increases electrode impedance and may displace and/or kill nearby neurons, leading to the reduced functionality or failure of the BCI. Ultimately, this results in diminished quality of care for patients who may require lifelong BCI intervention for treatment and hinders neuroscience research into long-term neuronal dynamics (e.g. learning, plasticity).

As such, there are several research thrusts which aim to design high-performance, high-density electrode arrays which mitigate gliosis to enable BCI to serve as a long-term treatment modality for some of the most debilitating, life-long neurological injuries and diseases. Key design factors that have been identified to limit gliosis severity include: modifying the mechanical properties of the electrode to be similar to that of brain tissue by modifying the material composition of the device; minimizing the extent of implantation-induced damage, particularly vascular damage, by minimizing the footprint of the device and using methods for precision implantation; and incorporating coatings and mechanisms for release of bioactive molecules which actively modulate the inflammatory reactivity of relevant immune effector cells.

Hydrogels and electrically active organic species are two classes of materials which have found widespread application in the electrode design toolbox for their ability to improve electrode properties in an attempt to mitigate the negative effects of gliosis on long-term BCI performance Hydrogels are a class of water-soluble or hydrophilic polymers that have demonstrated an ability to dampen the neuroinflammatory response and improve electrode recording outcomes. More specifically, hydrogel coatings, such as poly(2-hydroxyethyl methacrylate) (PHEMA), on the surface of electrodes lessen tissue strain induced by the mechanical mismatch between hard silicon/metallic electrodes and nervous tissue with Young's modulus >10 Gpa versus about 10 kPa, respectively. These studies demonstrate that the reduction in mechanical tissue strain scales with hydrogel thickness. However, thicker hydrogels also have the potential to displace neuronal cell bodies away from electrode recording sites, thus reducing signal quality, and do little to actively modulate the neuroinflammatory response beyond serving as sink for pro-inflammatory cytokines. Coatings composed of organic conductors, including conductive polymers such as the highly studied poly(3,4-ethylenedioxy thiophene) (PEDOT), carbon nanotubes (CNT), and graphene (GR), have been demonstrated improve the electrical connection between the electrode and neurons. However, when implemented in isolation, these improvements are still overshadowed by the thickness and density of the glial scar which may still limit the formation of an effective electrical interface over extended implant durations.

Conductive hydrogels are another class of materials which have been applied to neural electrodes because they combine the best features of both components: the tunable mechanical and swelling properties of hydrogels and the highly conductive nature of organic conductors. Many research efforts involve the coating of conductive hydrogels onto the surface of classical electrode arrays to reduce the mechanical mismatch at the device-tissue interface while retaining high signal quality for recording and/or stimulating purposes. Coating of classical electrode arrays with conductive hydrogels typically involves a 2-step process in which the hydrogel is coated onto the array first and then conductive polymers are grown from the electrode contact surface throughout the hydrogel matrix in a subsequent step via controlled electrochemical polymerization.

Conductive hydrogel coatings have demonstrated major improvements over non-coated classical electrode arrays and thus provided evidence for the positive impact these materials have on limiting the negative effects of gliosis. However, the size of the electrode footprint (i.e. cross-sectional area) is still a major issue for the damage it causes upon implantation which is made worse with a thicker hydrogel coating. Furthermore, the stiff substrate beneath the gel coating still may cause tissue damage over prolonged periods of brain micromotion and thus cause BCI failure over clinically relevant time frames (i.e., years to decades).

A shift in the field of neural engineering has been toward modifying the substrate material onto which electrode traces and contacts are fabricated toward more flexible and/or elastic materials such as parylene-C (about 2 GPa), poly(imide) (PI; about 1 GPa), poly(dimethyl siloxane) (PDMS; about 1 MPa) and most recently perfluoropoly(ether)-dimethacrylate (PFPEDMA; about 30 kPa). These modifications also coincide with replacement of the metallic electrical components such as gold or platinum with softer organic composite materials such as conductive hydrogels. It should be noted that, while there have been significant advances in this area, none have yet demonstrated robust neural electrode function in vivo for clinically relevant time scales. As such, the long-term performance reliability of neural microelectrodes remains an outstanding issue in the field that necessitates the design of new methods which may enable the fabrication of soft, high density electrode arrays which may limit implant-induced gliosis over chronic time scales. Despite the advances in the development of fabrication techniques for the preparation of soft, conductive materials as noted above, a need exists for new and improved methods for the synthesis of soft, conductive materials via high throughput fabrication techniques. These new and improved fabrication methods will have broad applicability to conductive materials and soft electronic devices useful as immunomodulatory neuroelectronic interfaces, such as neural electrodes. The present invention seeks to fulfill this need and provides further related advantages.

### SUMMARY OF THE INVENTION

The present invention provides methods for making conductive hydrogels, liquid resins for preparing conductive hydrogels, conductive hydrogels prepared by the methods or from the liquid resins, and electrodes comprising the conductive hydrogels.

In one aspect, the invention provides a method for making a conductive hydrogel. In the method, one or more polymerizable hydrogel-forming agents are photochemically polymerized in the presence of a polymerizable conductive monomer to provide a conductive hydrogel. In the method, the polymerizable hydrogel-forming agents comprise hydrogel-forming monomers and a crosslinking agent.

In certain embodiments, the invention provides a method for making a conductive hydrogel. The method comprises irradiating a solution comprising:

(a) a polymerizable hydrogel-forming monomer or a polymerizable hydrogel-forming macromer;

(b) a crosslinking agent effective to react with the monomer or macromer to provide a crosslinked hydrogel;

(c) a photoinitiator effective to initiate photopolymerization of the monomer or macromer and the crosslinking agent;

(d) a polymerizable conductive monomer;

(e) an oxidative initiator effective to initiate polymerization of the polymerizable conductive monomer; and

(f) a solvent effective to solubilize (a)-(e),

wherein one or more of the polymerizable hydrogel-forming monomer, polymerizable hydrogel-forming macromer, the crosslinking agent, the photoinitiator, or the oxidative initiator include an anionic or cationic moiety,

wherein the solution is irradiated with light having a wavelength effective to initiate polymerization of the polymerizable hydrogel-forming monomer or macromer and polymerization of the polymerizable conductive monomers, and

wherein the solution is irradiated at a temperature and for a time sufficient to provide a conductive hydrogel.

In another aspect of the invention, a liquid resin is provided. In certain embodiments, the liquid resin comprises:

(a) a polymerizable hydrogel-forming monomer or a polymerizable hydrogel-forming macromer;

(b) a crosslinking agent effective to react with the monomer or macromer to provide a crosslinked hydrogel;

(c) a photoinitiator effective to initiate photopolymerization of the monomer or macromer and the crosslinking agent;

(d) a polymerizable conductive monomer;

(e) an oxidative initiator effective to initiate polymerization of the polymerizable conductive monomer; and

(f) a solvent effective to solubilize (a)-(e),

wherein one or more of the polymerizable hydrogel-forming monomer, polymerizable hydrogel-forming macromer, the crosslinking agent, the photoinitiator, or the oxidative initiator include an anionic or cationic moiety.

In a related aspect, the invention provides a method for making a conductive object or conductive pattern. In certain embodiments, the liquid resin comprises:

(a) 3D printing, micropatterning, or photolithographically or stereolithography patterning a liquid resin as described herein to provide a printed or patterned resin; and

(b) irradiating the printed or patterned resin to provide a conductive object or conductive pattern.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIGS. 1A-1D compare scanning electron micrographs of conductive hydrogels. FIGS. 1A and 1B are low (1A) and high (1B) magnification scanning electron micrographs of precision porous scaffolds prepare by standard two-pot conductive hydrogel synthesis process and demonstrate poor spatial control of PEDOT:PSS particle growth. FIGS. 1C and 1D are low (1C) and high (1D) magnification micrographs of precision porous scaffolds of the invention prepared by the one-pot conductive hydrogel synthesis method of the invention and show a precision porous network with no adverse particle formation. Scale bars are 200 μm (1A), 10 μm (1B), 100 μm (1C), 10 μm (1D).

FIGS. 2A-2C compare images of representative conductive hydrogels of the invention with HEMA- and PVA-based hydrogels. FIGS. 2A and 2B compare hydrogels prepared using different polymerization times: polymerization time series of monomeric HEMA-based gels (2A) and macromeric PVA-based gels (2B) prepared by the one-pot conductive hydrogel synthesis method of the invention. FIG. 2C illustrates fabricated discs of both gel systems (with and without EDOT in the respective pre-gel solutions) at their swelling equilibrium state in PBS (pH 7.4).

FIGS. 3A-3D compare scanning electron micrographs of representative conductive hydrogels (H50C-2.5E and PVA-2.5E) of the invention with corresponding control non-conductive hydrogels (H50C and PVA): H50C (3A), H50C-2.5E (3B), PVA (3C), and PVA-2.5E (3D).

FIGS. 4A-4D compare the hydration, mechanical, and electrochemical properties of representative conductive hydrogels of the invention. FIG. 4A compares the swelling ratio of HEMA and PVA gel systems. FIG. 4B compares mechanical properties (compression modulus) of HEMA and PVA gel systems measured via uniaxial compression at 0.1 mm/min compression speed. FIG. 4C compares representative electrical impedance spectra (EIS) of HEMA and PVA systems. FIG. 4D compares impedance magnitude of HEMA and PVA systems. Quantification of impedance magnitude at 1 kHz stimulation frequency are statistically significant when comparing PEDOT-containing and control gels in the same system (asterisks), and between systems (H50C vs. PVA, solid bars, single hash; H50C-2.5E vs. PVA-2.5E, striped bars, double hash). Black dotted line is impedance of platinum control at 1 kHz.

FIGS. 5A-5D compare the hydration, mechanical, conductivity, and vibrational spectroscopic properties of representative conductive hydrogels of the invention. FIG. 5A compares the swelling ratio of four groups of hydrogels (HG50S, HG25S, HG50C, and HG25C). FIG. 5B compares the mechanical properties (compression modulus) of the four groups, which follows similar trends in the swelling ratio. FIG. 5C compares conductivity of the gels measured via a standard 2-probe technique. FIG. 5D compares the Raman spectra of the region containing PEDOT peaks (1170-1630 cm⁻¹) demonstrating differences in peak shift across the groups as a measure of the degree of doping on the PEDOT backbone. The insert plots differences in peak shift across the 4 gel samples.

FIGS. 6A-6F compare the hydration, mechanical, conductivity, vibrational spectroscopic, and electrical impedance properties of representative conductive hydrogels of the invention. The properties demonstrate that the concentration of EDOT in the pre-gel mixture has an impact on final gel properties. FIG. 6A compares the swelling ratio of two groups of hydrogels (HG50SC, HG25SC) as a function of EDOT concentration in the pre-gel mixture. FIG. 6B compares the mechanical compression of two groups of hydrogels (HG50SC, HG25SC) as a function of EDOT concentration in the pre-gel mixture. FIG. 6C compares the conductivity of two groups of hydrogels (HG50SC, HG25SC) as a function of EDOT concentration in the pre-gel mixture. FIG. 6D compares the Raman spectra of two groups of hydrogels (HG50SC, HG25SC) as a function of EDOT concentration in the pre-gel mixture. FIG. 6E compares the electrochemical impedance spectra of five groups of hydrogels (HG50SC-2.5E, HG50SC-5E, HG50SC-7.5E, HG25SC-2.5E, HG25SC-5E). FIG. 6F compares the electrochemical impedance spectra of two groups of hydrogels (HG50SC, HG25SC) as a function of EDOT concentration in the pre-gel mixture.

FIG. 7 is an image of a representative conductive hydrogel electrode of the invention. The electrode (or array of electrodes) was prepared by the one-pot photochemical synthesis method of the invention via photolithography.

### DETAILED DESCRIPTION OF THE INVENTION

The present invention provides methods for making a conductive hydrogel, comprising photochemically polymerizing polymerizable hydrogel-forming agents in the presence of a polymerizable conductive monomer to provide a conductive hydrogel; liquid resins for making a conductive hydrogel comprising photochemically polymerizable hydrogel-forming agents and polymerizable conductive monomers; conductive hydrogels prepared by the methods or from the liquid resins; and electrodes comprising the conductive hydrogels.

In one aspect, the present invention provides a method for making conductive hydrogels. In the method, the conductive hydrogel is made by photochemically polymerizing polymerizable hydrogel-forming agents in the presence of a polymerizable conductive monomer to provide a conductive hydrogel.

In one embodiment of the method, a one-pot method is provided. As used herein, the term “one-pot method” refers to a method for preparing a conductive hydrogel from a single solution in one step. In the method, a solution that includes conductive hydrogel-forming components is irradiated. The solution comprises:

(a) a polymerizable hydrogel-forming monomer or a polymerizable hydrogel-forming macromonomer;

(b) a crosslinking agent effective to react with the monomer or macromer to provide a crosslinked hydrogel;

(c) a photoinitiator effective to initiate photopolymerization of the monomer or macromer and the crosslinking agent;

(d) a polymerizable conductive monomer;

(e) an oxidative initiator effective to initiate polymerization of the polymerizable conductive monomer; and

(f) a solvent effective to solubilize (a)-(e),

wherein one or more of the polymerizable hydrogel-forming monomer, polymerizable hydrogel-forming macromer, the crosslinking agent, the photoinitiator, or the oxidative initiator include an anionic or cationic moiety that serves as a dopant to facilitate conductive polymer formation and hydrogel electrical activity.

In certain embodiments, the polymerizable hydrogel-forming monomer or polymerizable hydrogel-forming macromer includes an anionic or cationic moiety that serves as the dopant.

In other embodiments, the oxidative initiator includes an anionic or cationic moiety that serves as the dopant.

In further embodiments, the polymerizable hydrogel-forming monomer or polymerizable hydrogel-forming macromer and the oxidative initiator include an anionic or cationic moiety that serve as the dopant.

In certain embodiments, none of the polymerizable hydrogel-forming monomer, the polymerizable hydrogel-forming macromer, the crosslinking agent, the photoinitiator, or the oxidative initiator include an anionic or cationic moiety. In these embodiments, the solution further includes a dopant that includes an anionic or cationic moiety that serves to facilitate conductive polymer formation and hydrogel electrical activity.

In the methods described herein, the solution is irradiated with light having a wavelength effective to initiate polymerization of the polymerizable hydrogel-forming monomer or macromer and polymerization of the polymerizable conductive monomers, and the solution is irradiated at a temperature and for a time sufficient to provide a conductive hydrogel.

A unique aspect of this methodology is the integration of the photoinitiator and oxidative initiator within the same reaction vessel. The combination provides an interaction such that the photolysis of the photoinitiator upon irradiation causes an acceleration of the lysis of the oxidative initiator that drastically increases the efficiency of the chemical polymerization of the conductive polymer. The result is a photochemical synthetic methodology for the rapid fabrication of conductive hydrogels.

The shape and size of the product conductive hydrogel can be varied by, for example, selecting the vessel (e.g., mold) in which the hydrogel-forming reaction is carried out. Other shapes and sizes of the conductive hydrogel can be obtained by other fabrication techniques, such as those described herein.

Representative photoinitiators include 2,2-dimethoxy-1,2-diphenylethanone (IRGACURE 651) and 2-hydroxy-4′-(2-hydroxyethoxy)-2-methylpropiophenone (IRGACURE 2959).

Representative oxidative initiators useful in the methods include persulfates (ammonium persulfate, APS; sodium persulfate, NaPS; potassium persulfate, KPS) and others like FeCl₃, with various trade-offs depending on the initiator chosen (efficiency, final conductivity, molar ratio of initiator:monomer required), which adds another layer of tunability to the synthesis method

In certain embodiments, the solvent is an alcohol. Representative alcohols include methanol, ethanol, isopropanol, poly(ethylene glycol) (PEG200), ethylene glycol (EG), and glycerol (Gly)). Other solvents include DI water, acetonitrile, and dimethylsulfoxide (DMSO).

The methods of the invention provide great flexibility in the nature of the conductive hydrogel to be prepared. The amount of each component can be varied to achieve the desired properties of the conductive hydrogel. The ratio of hydrogel:conductor:solvent may be varied and are not particularly dependent on specific relative amounts. The properties of the final hydrogel (hydration, mechanical, conductive) are thus tunable based on these relative ratios, with certain combinations outperforming others, as described herein.

In certain embodiments, the polymerizable hydrogel-forming agents comprise hydrogel-forming monomers and a crosslinking agent. The methods for making the hydrogels utilize polymerizable hydrogel-forming agents, such as radical polymerization-based monomers/macromers. In certain embodiments, the hydrogel-forming agents and crosslinking agents are acrylates and methacrylates.

In certain embodiments, the polymerizable hydrogel-forming monomers comprise 2-hydroxyethyl methacrylate (HEMA), glycerol methacrylate (GMA), methacrylic acid (MAA), and styrene sulfonate (SS), and the crosslinking agent is tetraethyleneglycol dimethacrylate (TEGDMA) or polyethylene glycol dimethacrylate (PEGDA).

In other embodiments, the polymerizable hydrogel-forming agents comprise hydrogel-forming macromers. A representative hydrogel-forming macromer is PVA-MA-Taurine.

In certain embodiments of the methods, the polymerizable conductive monomer is a polymerizable thiophene. In the practice of the invention, any conductive monomer that is susceptible to oxidative polymerization is effective. Representative conductive monomers include pyrroles and anilines, both of which are frequently polymerized with APS or other oxidative initiators. In one embodiment, the polymerizable thiophene is 3,4-ethylene dioxythiophene (EDOT).

The methods for making the conductive hydrogels of the invention include the use of a dopant (e.g., anionic, negatively charged groups, such as sulfonate or carboxylate groups; cationic, positively charged groups) that can be covalently immobilized to the hydrogel matrix during polymerization (e.g., via copolymerization with a sulfonate- or carboxylate-containing monomer or macromer, such as styrene sulfonate and methacrylic acid) to encourage the growth of the conductive polymer throughout the hydrogel network to form a true interpenetrating polymer network as opposed to having the conductive polymer grow in small disparate particles with the non-immobilized APS-based sulfate as dopant.

The choice of dopant (e.g., anionic or cationic) will depend on the doping mechanism of the conductive polymer used. For conductive polymers are n-type doped, a cationic dopant is used.

In certain embodiments, the dopant is incorporated into the hydrogel by polymerization of a hydrogel-forming monomer or macromer that includes an anionic (or cationic) group. In other embodiments, the dopant is incorporated into the hydrogel by including chemical species having an anionic (or cationic) group (e.g., crosslinking agent, photoinitiator, oxidative initiator, or other additional compound). In certain of these embodiments, the oxidative initiator (e g, ammonium persulfate, APS) can be used to both initiate the polymerization of the conductive polymer and remain in the hydrogel as an (non-immobilized) anionic dopant.

In certain embodiments, the methods for making conductive hydrogels further comprises the use of sphere-templated scaffold fabrication to produce the conductive hydrogel.

In another aspect on the invention, conductive hydrogels are provided. In certain embodiments, the conductive hydrogel of the invention is a conductive interpenetrating polymer network that includes a hydrogel and a conductive polymer. As used herein, the term “interpenetrating polymer network” refers to an intimate combination (i.e., network) of the hydrogel and the conductive polymers that results from the polymerizations of the hydrogel-forming polymerizable monomer and macromonomers that provide the hydrogel in the presence of the polymerization of the polymerizable conductive monomers that provide the conductive polymers.

It will be appreciated that in certain embodiments a covalent interaction between the hydrogel network and conductive polymers may form during formation of the conductive hydrogel. It will be appreciated that in addition to interpenetrating polymer networks, the hydrogels of the invention can include covalent interactions between the hydrogel and conductive polymer components of the hydrogel.

The conductive hydrogel (or conductive interpenetrating polymer network) has advantageous hydration, compression modulus, and electrical impedance, as described herein.

In a further aspect, the invention provides conductive hydrogel-containing devices.

In certain embodiments, the invention provides an electrode comprising a conductive hydrogel, as described herein. The electrode may be an implantable electrode (i.e., within a subject, such as within a tissue or an organ, such as the brain or heart). The electrode may be positioned on the surface of a subject (e.g., on the skin of a subject) or on the surface of a tissue or organ.

In other embodiments, the invention provides a sensor comprising a conductive hydrogel, as described herein.

In certain embodiments, the invention provides a medical device comprising the electrode or sensor.

In certain embodiments, the invention provides a neuroelectronic interface comprising the electrode or sensor.

In another aspect, the invention provides a conductive hydrogel-forming resin useful for forming an object (having a desired shape) or a pattern on a surface by techniques known in the art (e.g., 3D printing, micropatterning, photolithography, stereolithography).

In one embodiment, the invention provides a liquid resin for 3D printing, micropatterning, photolithography, or stereolithography, comprising:

(a) a polymerizable hydrogel-forming monomer or a polymerizable hydrogel-forming macromer;

(b) a crosslinking agent effective to react with the monomer or macromer to provide a crosslinked hydrogel;

(c) a photoinitiator effective to initiate photopolymerization of the monomer or macromer and the crosslinking agent;

(d) a polymerizable conductive monomer;

(e) an oxidative initiator effective to initiate polymerization of the polymerizable conductive monomer; and

(f) a solvent effective to solubilize (a)-(e),

wherein one or more of the polymerizable hydrogel-forming monomer, polymerizable hydrogel-forming macromer, the crosslinking agent, the photoinitiator, or the oxidative initiator include an anionic or cationic moiety that serves as a dopant to facilitate conductive polymer formation and hydrogel electrical activity.

In certain embodiments, the polymerizable hydrogel-forming monomer or polymerizable hydrogel-forming macromer includes an anionic or cationic moiety that serves as the dopant.

In other embodiments, the oxidative initiator includes an anionic or cationic moiety that serves as the dopant.

In further embodiments, the polymerizable hydrogel-forming monomer or polymerizable hydrogel-forming macromer and the oxidative initiator include an anionic or cationic moiety that serve as the dopant.

In certain embodiments, none of the polymerizable hydrogel-forming monomer, the polymerizable hydrogel-forming macromer, the crosslinking agent, the photoinitiator, or the oxidative initiator include an anionic or cationic moiety to serve as a dopant to facilitate conductive polymer formation and hydrogel electrical activity. In these embodiments, the liquid resin further includes a dopant that includes an anionic or cationic moiety that serves to facilitate conductive polymer formation and hydrogel electrical activity.

In a related embodiment, the invention provides a method for making a conductive object or conductive pattern, comprising 3D printing, micropatterning, photolithography, or stereolithography patterning the liquid resin to provide a printed or patterned resin; and irradiating the printed or patterned resin to provide a conductive object or conductive pattern. Conductive objects and patterns prepared by the method are also provided.

The following is a description of representative conductive hydrogels of the invention, methods for making the conductive hydrogels, and representative uses of the conductive hydrogels.

The preparation of representative conductive hydrogels of the invention is described in Example 1.

Monomer and Macromer Hydrogel Systems

As noted above, the conductive hydrogels of the invention can be prepared from monomers or macromers. Table 1 summarizes pre-gel compositions for representative monomer and macromer hydrogel systems.

TABLE 1
Pre-gel compositions for experiments investigating impact of a
monomer vs. macromer hydrogel system on materials produced
via the one-pot synthesis method.

| | Hydrogel | | Dopant | |
|---|---|---|---|---|
| | Concen- | | Concen- | EDOT^{a)} |
| | tration | Dopant | tration | Concentration |
| Gel Name | [%] | Selection | [mol/L] | [%]^{b)} |
| H50C | 50^{b)} | AA^{d)} (—COO⁻) | 0.25 | 0 |
| H50C-2.5E | 50^{b)} | AA^{d)} (—COO⁻) | 0.25 | 2.5 |
| PVA | 8^{c)} | Taurine (—SO₃⁻) | 0.1 | 0 |
| PVA-2.5E | 8^{c)} | Taurine (—SO₃⁻) | 0.1 | 2.5 |

^{a)}3,4-ethylenedioxy thiophene; ^{b)}volume/volume; ^{c)}weight/volume; ^{d)}acrylic acid

Conductive Hydrogel Morphology

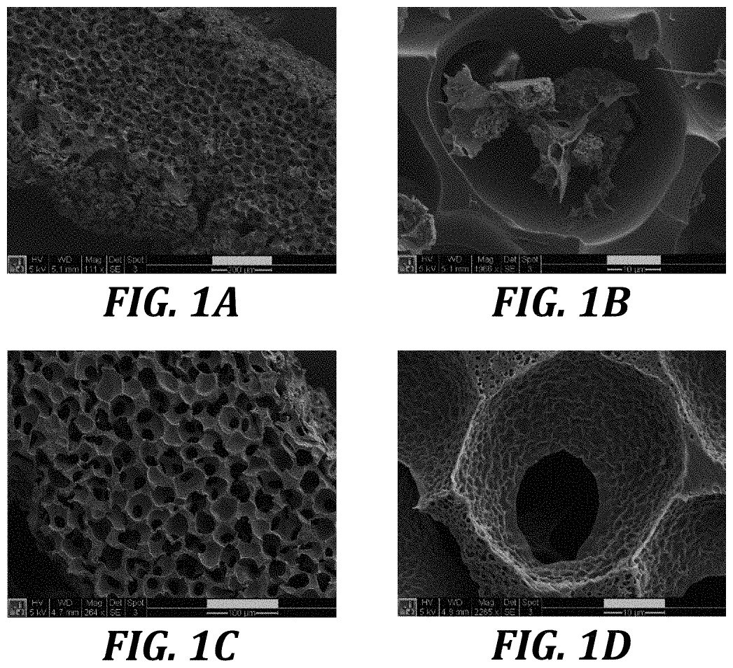

Scanning electron micrographs of a representative conductive hydrogel of the invention prepared by the one-pot synthesis method of the invention are compared to conventional two-pot synthesized hydrogels are shown in FIGS. 1A-1D. FIGS. 1A and 1B are low (1A) and high (1B) magnification scanning electron micrographs of precision porous scaffolds prepare by standard two-pot conductive hydrogel synthesis process and demonstrate poor spatial control of PEDOT:PSS particle growth. FIGS. 1C and 1D are low (1C) and high (1D) magnification micrographs of precision porous scaffolds of the invention prepared by the one-pot conductive hydrogel synthesis method of the invention and show a precision porous network with no adverse particle formation. The high magnification image also demonstrates a finer microstructure imparted to the pore surfaces and walls on the order of 1 μm throughout the material which may promote cellular adhesion/migration and/or nutrient diffusion throughout the scaffold. Scale bars are 200 μm (1A), 10 μm (1B), 100 μm (1C), 10 μm (1D).

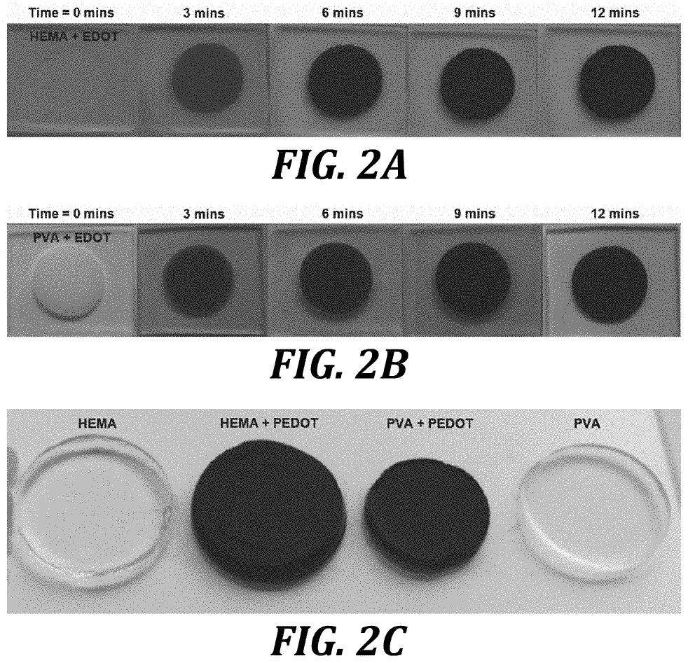

FIGS. 2A-2C compare images of representative conductive hydrogels of the invention with HEMA- and PVA-based hydrogels. FIGS. 2A and 2B compare hydrogels prepared using different polymerization times: polymerization time series of monomeric HEMA-based gels (2A) and macromeric PVA-based gels (2B) prepared by the one-pot conductive hydrogel synthesis method of the invention. The process enables rapid photochemical polymerization of PEDOT alongside the hydrogel component, but also increases gelation time from 3 minutes to 12 minutes. FIG. 2C illustrates fabricated discs of both gel systems (with and without EDOT in the respective pre-gel solutions) at their swelling equilibrium state in PBS (pH 7.4).

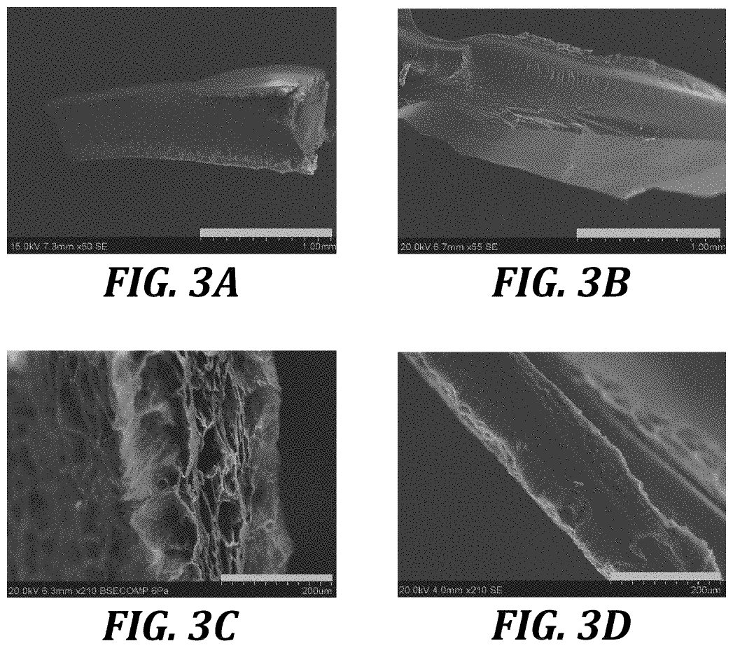

FIGS. 3A-3D compare scanning electron micrographs of representative conductive hydrogels (H50C-2.5E and PVA-2.5E) of the invention with corresponding control non-conductive hydrogels (H50C and PVA): H50C (3A), H50C-2.5E (3B), PVA (3C), and PVA-2.5E (3D). PVA gels are the least dense as can be seen by the large porous network throughout the gel cross section (3C). H50C gels also show some porosity on the top and bottom surfaces. Both H50C-2.5E and PVA-2.5E gels are completely “filled” but no PEDOT particles are observable at the magnifications examined

Conductive Hydrogel Hydration, Mechanical, and Electrochemical Properties

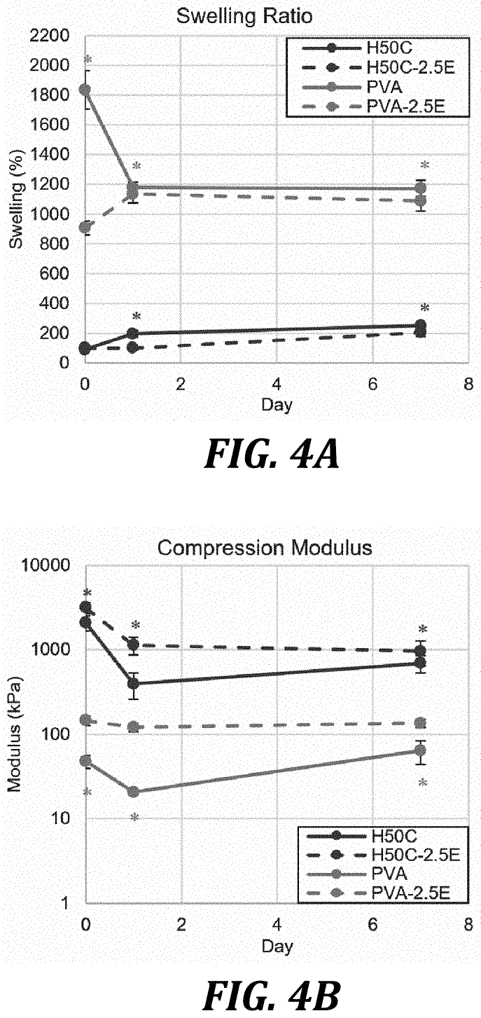

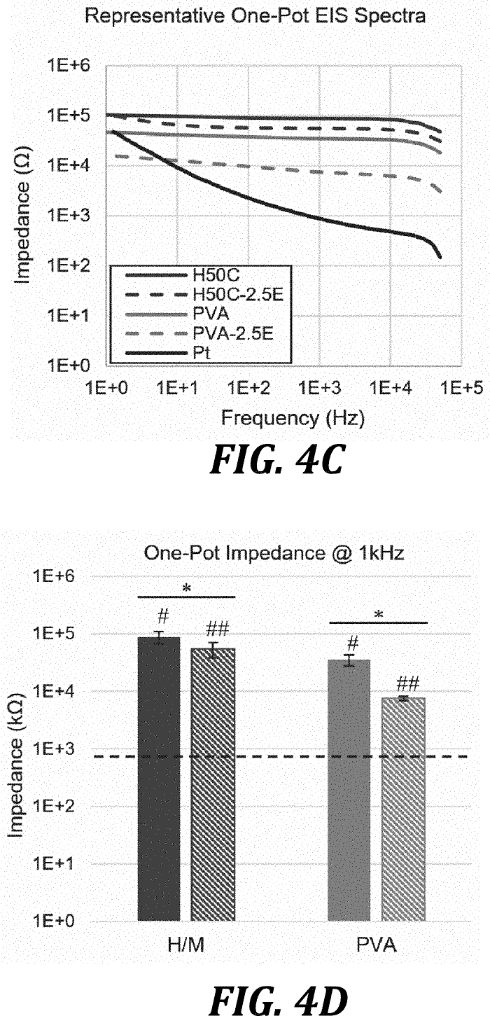

The hydration, mechanical, and electrochemical properties of representative conductive hydrogels of the invention are compared in FIGS. 4A-4D.

FIG. 4A compares the swelling ratio of HEMA and PVA gel systems. PVA gels experience a significant increase in swelling compared to HEMA gels, consistent with gel densities used for each system. Both PEDOT-containing gels, H50C-2.5E and PVA-2.5E, swelled slightly less than their respective controls, H50C and PVA. Though minor, these differences were statistically significant for all gels (color coded asterisks) except on Day 0 for H50C vs. H50C-2.5E gels.

FIG. 4B compares mechanical properties (compression modulus) of HEMA and PVA gel systems measured via uniaxial compression at 0.1 mm/min compression speed. The HEMA systems were much stiffer than PVA systems, also consistent with gel densities used for each system. Additionally, differences between PEDOT-containing and control gels within both systems demonstrated statistically significant differences across all days (coded asterisks). This suggests interactions of PEDOT and hydrogel networks upon experiencing compressive strain.

FIG. 4C compares representative electrical impedance spectra (EIS) of

HEMA and PVA systems and demonstrates that a reduction in impedance occurs when PEDOT is incorporated into the system. Improvements in the impedance characteristics of PVA-2.5E are disproportionately larger than H50C-2.5E, as compared to their respective controls, indicating gel density is a major factor impacting gel electrical properties along with potential other factors such as dopant quality.

FIG. 4D compares impedance magnitude of HEMA and PVA systems.

Quantification of impedance magnitude at 1 kHz stimulation frequency are statistically significant when comparing PEDOT-containing and control gels in the same system (asterisks), and between systems (H50C vs. PVA, solid bars, single hash; H50C-2.5E vs. PVA-2.5E, striped bars, double hash). Black dotted line is impedance of platinum control at 1 kHz.

Gel Density and Dopant Quality The impact of gel density and dopant quality on conductive hydrogel properties were investigated to in a representative system (HEMA monomer). Table 2 summarizes pre-gel compositions for investigating gel density and dopant quality in representative conductive hydrogels of the invention.

TABLE 2
Pre-gel composition for experiments investigating the impact of
monomer concentration and dopant chemistry on final gel properties.
The hydrogel component used for these experiments was
comprised of a 4:1 mixture of HEMA:GMA.

| | Monomer | | Dopant | EDOT^{a)} |
|---|---|---|---|---|
| Gel | Concentration | Dopant | Concentration | [%, |
| Name | [%, v/v] | Selection | [mol/L] | v/v] |
| HG50S | 50 | AMPS^{b)} (—SO₃⁻) | 0.1 | 0 |
| HG50S-2.5E | 50 | AMPS^{b)} (—SO₃⁻) | 0.1 | 2.5 |
| HG25S | 25 | AMPS^{b)} (—SO₃⁻) | 0.1 | 0 |
| HG25S-2.5E | 25 | AMPS^{b)} (—SO₃⁻) | 0.1 | 2.5 |
| HG50C | 50 | AA^{c)} (—COO⁻) | 0.1 | 0 |
| HG50C-2.5E | 50 | AA^{c)} (—COO⁻) | 0.1 | 2.5 |
| HG25C | 25 | AA^{c)} (—COO⁻) | 0.1 | 0 |
| HG25C-2.5E | 25 | AA^{c)} (—COO⁻) | 0.1 | 2.5 |

^{a)}3,4-ethylenedioxy thiophene; ^{b)}2-acrylamido-2-methylpropane sulfonic acid; ^{c)}acrylic acid

In Table 2, HG refers to HEMA/glycerol methacrylate (GMA), a copolymer of the two monomers at 80/20 ratio Immobilized sulfonic acid moieties originated from AMPS monomers. Immobilized carboxylic acid moieties originated from acrylic acid monomers.

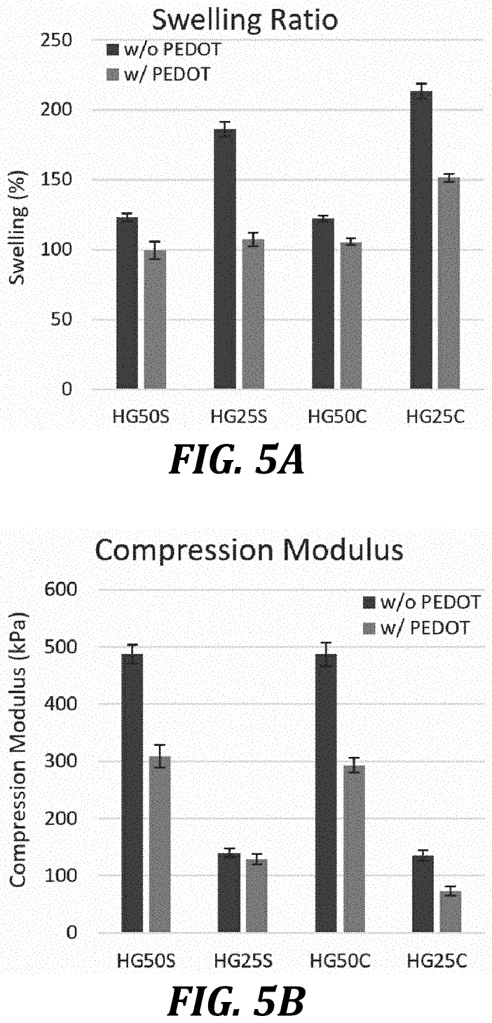

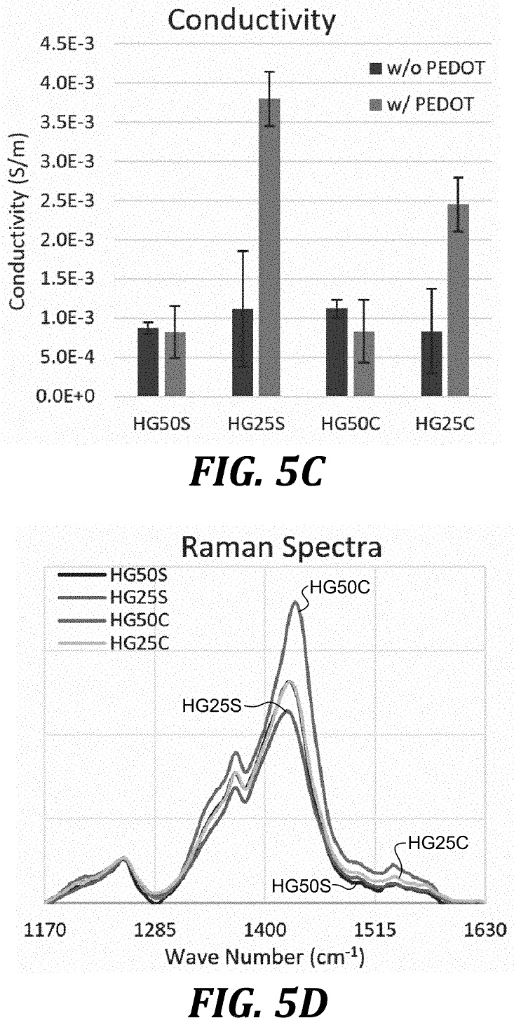

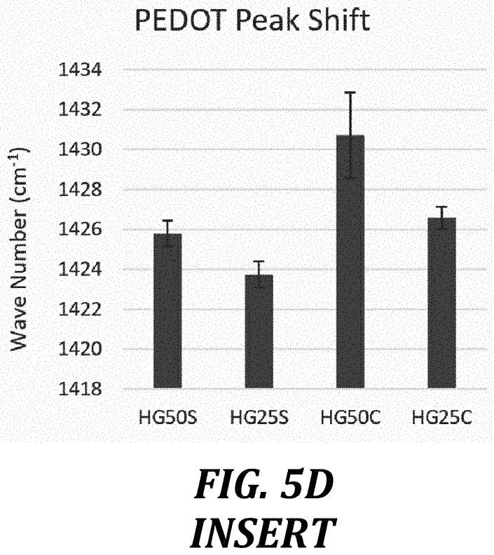

The hydration, mechanical, conductivity, and vibrational spectroscopic properties of representative conductive hydrogels of the invention are compared in FIGS. 5A-5D.

FIG. 5A compares the swelling ratio of four groups of hydrogels (HG50S, HG25S, HG50C, and HG25C) and demonstrates a reduction in swelling for PEDOT-containing gels compared to their respective controls, consistent with previous findings. Both groups of gels with 50% gel density (HG50S, HG50C) demonstrated identical swelling patterns for both PEDOT-containing and control gels. Gels in the 25% gel density groups (HG25S, HG25C) demonstrate increased swelling ratios compared to 50% gel groups, although PEDOT-containing gels in the group with 25% gel density and sulfonic acid dopant (HG25S-2.5E) showed disproportionate decrease in swelling compared to its control (HG25S), suggesting differences in interactions between the PEDOT chains and hydrogel matrix in this group compared to other groups.

FIG. 5B compares the mechanical properties (compression modulus) of the four groups, which follows similar trends in the swelling ratio. PEDOT-containing groups demonstrated a reduction in compression moduli compared to their respective controls. This may have occurred as a result of differences in UV exposure setup used between these two sets of experiments, or perhaps as a result of the inclusion of glycerol methacrylate in the pre-gel composition for these experiments.

FIG. 5C compares conductivity of the gels measured via a standard 2-probe technique and demonstrate no change in conductivity between PEDOT-containing and control gels for the 50% gel density groups, which suggests that the PEDOT may have been overoxidized or otherwise limited in producing an electrical connection throughout the gel. The 25% gel density groups both showed an increase in conductivity compared to their respective controls, with the sulfonic acid-doped group (HG25S-2.5E) outperforming the carboxylic acid-doped (HG25C-2.5E). This provides evidence that the immobilized sulfonic acid moieties are providing a better doping environment for the PEDOT than the immobilized carboxylic acid moieties, perhaps as a result of the differences in pKₐ of the respective acids.

FIG. 5D compares the Raman spectra of the region containing PEDOT peaks (1170-1630 cm⁻¹) and demonstrates differences in peak shift across the groups as a measure of the degree of doping on the PEDOT backbone. The group of gels which performed the best from a conductivity stand-point, HG25S-2.5E, demonstrated a shift to lower wave number, which has been associated with increases in chain linearity and chain-chain hopping as a result of improvements to PEDOT doping (see 5D insert). The inset plots differences in peak shift across the 4 gel samples.

EDOT Pre-Gel Concentration

EDOT pre-gel concentration was investigated for the purpose of improving the conductivity and electrical impedance of the gels in anticipation of potential application to neural microelectrodes. In these experiments, EDOT concentration in the pre-gel solution was varied from 2.5% to 7.5% across both 50% and 25% gel density groups. Both sulfonic acid and carboxylic acid dopants were immobilized in each gel as it was found that doing so enabled gelation of 25% gel samples with 5% EDOT whereas gelation did not occur in 25% gel samples with >2.5% EDOT with only sulfonic acid dopants. Gelation of 25% gel density samples with 7.5% EDOT did not occur, regardless of these changes.

Table 3 summarizes pre-gel composition EDOT concentration and gel density and dopant for representative conductive hydrogels of the invention.

TABLE 3
Pre-gel Compositions for experiments investigating the impact of
pre-gel EDOT concentration on final gel properties. The hydrogel
component used for these experiments was comprised of a 4:1
mixture of HEMA:GMA.

| | Gel | | | |
|---|---|---|---|---|
| | Density | | Dopant, | EDOT^{a)} |
| Gel Name | [%, v/v] | Dopant Selection | [mol/L] | [%, v/v] |
| HG25SC-2.5E | 25 | AMPS^{b)} & AA^{c)} | 0.2 | 2.5 |
| | | (—SO₃⁻ & —COO⁻) | | |
| HG25SC-5E | 25 | AMPS^{b)} & AA^{c)} | 0.2 | 5.0 |
| | | (—SO₃⁻ & —COO⁻) | | |
| HG50SC-2.5E | 50 | AMPS^{b)} & AA^{c)} | 0.2 | 2.5 |
| | | (—SO₃⁻ & —COO⁻) | | |
| HG50SC-5E | 50 | AMPS^{b)} & AA^{c)} | 0.2 | 5.0 |
| | | (—SO₃⁻ & —COO⁻) | | |
| HG50SC-7.5E | 50 | AMPS^{b)} & AA^{c)} | 0.2 | 7.5 |
| | | (—SO₃⁻ & —COO⁻) | | |

^{a)}3,4-ethylenedioxy thiophene; ^{b)}2-acrylamido-2-methylpropane sulfonic acid; ^{c)}acrylic acid

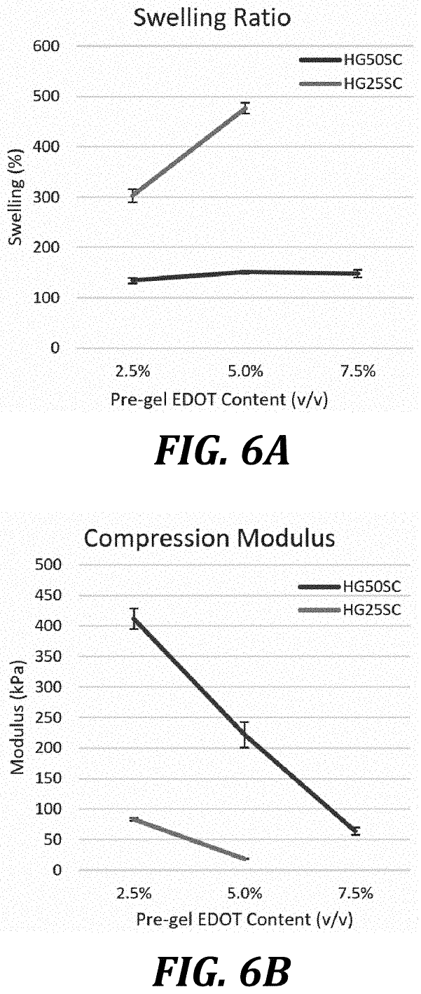

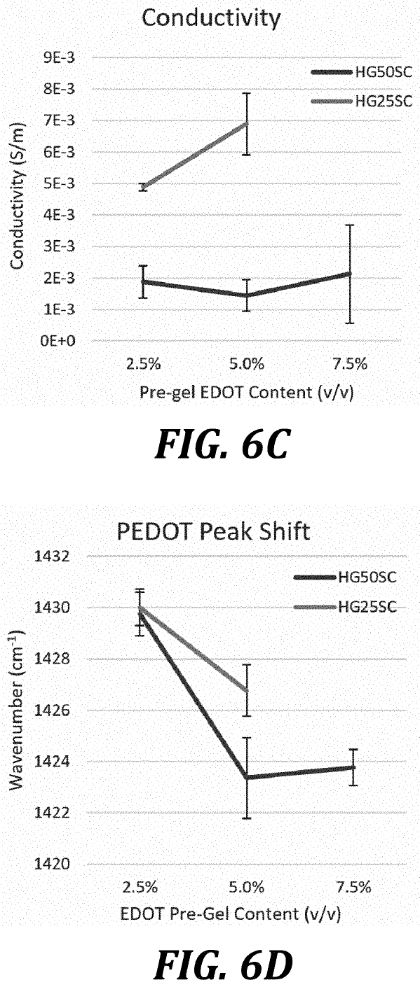

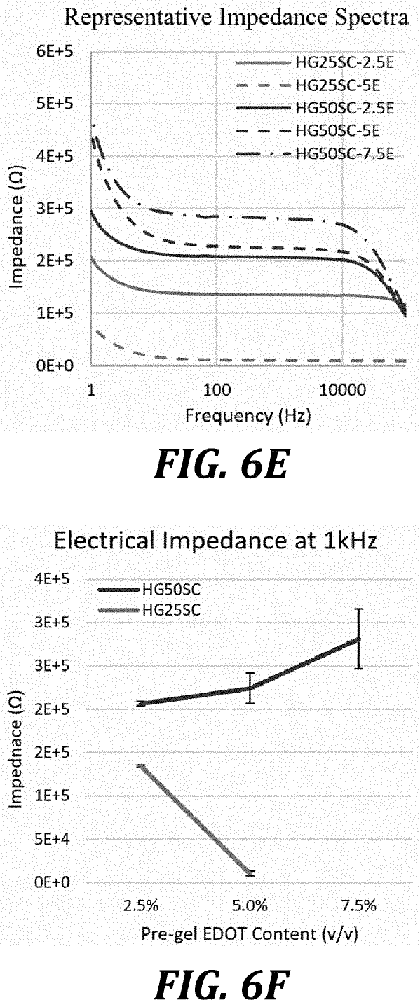

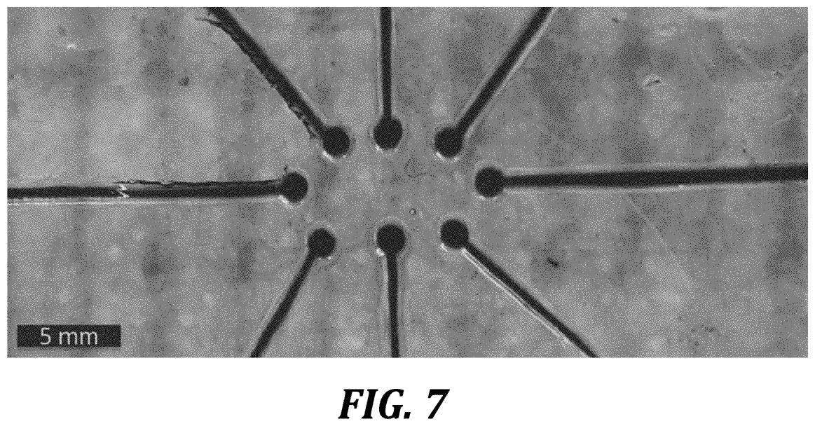

The hydration, mechanical, conductivity, vibrational spectroscopic, and electrical impedance properties of representative conductive hydrogels of the invention are compared in FIGS. 6A-6F. The properties demonstrate that the concentration of EDOT in the pre-gel mixture has an impact on final gel properties.

FIG. 6A compares the swelling ratio of two groups of hydrogels (HG50SC, HG25SC) as a function of EDOT concentration in the pre-gel mixture. Increasing EDOT content induces an increasing in swelling ratio only in gels with a low pre-gel monomer concentration. The differences observed between the groups with low and high pre-gel monomer concentration indicate changes may be occurring in the crosslinking process during gelation once the pre-gel monomer concentration passes below some threshold.

FIG. 6B compares the mechanical compression of two groups of hydrogels (HG50SC, HG25SC) as a function of EDOT concentration in the pre-gel mixture.

Increasing EDOT content causes a monotonic decrease in compression modulus regardless of pre-gel monomer concentration. The linear decrease in compression modulus for the higher pre-gel monomer concentration groups observed in conjunction with the swelling data in (a) provide additional evidence that the crosslinking is similar within the gels as the EDOT concentration increases, and that the reduction in compression modulus is a result of lower monomer conversion producing a less dense gel. As such, the reduction in compression modulus with the low pre-gel monomer concentration groups is likely a combination of reduced monomer conversion and changes in the crosslinking within the gel. HG25SC-5.0E gel produced a compression modulus of about 18 kPa which is very close to the mechanical properties of brain tissue (about 10 kPa).

FIG. 6C compares the conductivity of two groups of hydrogels (HG50SC, HG25SC) as a function of EDOT concentration in the pre-gel mixture. The conductivity of the gels followed similar trends to the swelling data in (a). The improvements in conductivity of the gels with low pre-gel monomer concentration when exposed to a DC voltage were likely due to a combination of an increase in swelling which would improve the ionic conductivity of the gels, and an increase in PEDOT content which would improve both the ionic and electronic conductivity of the gels. Gels with high pre-gel monomer concentration showed no improvement in conductivity which suggests any PEDOT in the gels is not sufficiently forming an interconnected network to pass charge through the bulk of the gel.

FIG. 6D compares the Raman spectra of two groups of hydrogels (HG50SC, HG25SC) as a function of EDOT concentration in the pre-gel mixture. Increasing pre-gel EDOT concentration causes a shift of the PEDOT peak to lower wave numbers, indicating a subsequent improvement to PEDOT doping. However, in light of conductivity data, the improvements to doping realized by increasing pre-gel EDOT concentration do not translate directly to improvements in conductivity in the presence of a DC voltage for gels groups with high pre-gel monomer concentration. This may be because a greater proportion of the PEDOT chains are in a more doped state but the edges of the PEDOT particles are significantly overoxidized or insulated by the hydrogel matrix such that no improvements in the PEDOT particle structure may have an impact on the bulk conductivity of the gel. This indicates that degree of doping is not directly correlated to bulk electrical properties of the resulting gels.

FIG. 6E compares the electrochemical impedance spectra of five groups of hydrogels (HG50SC-2.5E, HG50SC-5E, HG50SC-7.5E, HG25SC-2.5E, HG25SC-5E). Impedance of the gels demonstrates another bifurcation of the impact of pre-gel EDOT concentration on the resulting gel properties. Increasing pre-gel EDOT concentration in gel groups with low pre-gel monomer concentration shows a significant reduction in electrical impedance magnitude of the material that is in agreement with conductivity and doping data. Conversely, increasing pre-gel EDOT concentration in gel groups with high pre-gel monomer concentration causes an increase in impedance magnitude which provides additional evidence that the PEDOT network forming in these gels is increasingly more insulative than conductive.

FIG. 6F compares the electrochemical impedance spectra of two groups of hydrogels (HG50SC, HG25SC) as a function of EDOT concentration in the pre-gel mixture. Quantification of impedance magnitude across the gels at 1 kHz stimulation frequency, a useful frequency for recording neuronal activity, demonstrates the magnitude of the differences observed between the material types tested.

The conductive hydrogels of the invention can be used in a variety of applications. As noted above, the conductive hydrogel of the invention can be used as an electrode. FIG. 7 is an image of a representative conductive hydrogel electrode of the invention. The electrode (or array of electrodes) was prepared by the one-pot photochemical synthesis method of the invention via photolithography. The liquid pre-gel composition HG25SC-3.0E was deposited in a thin layer on a glass slide and covered with a custom-made mask followed by exposure to UV radiation with a broad-spectrum UV light for 5 minutes. Removal of the mask left the photochemically polymerized traces, demonstrating the effectiveness of the application of the one-pot photochemical synthesis method to photolithographic techniques.

The conductive hydrogels of the invention described herein expand the neural electrode design toolbox. The invention provides facile methods for the synthesis of soft, conductive materials with broad applicability to various high throughput fabrication techniques.

The following examples are provided for the purpose of illustrating, not limiting, the invention.

### EXAMPLES

### Example 1

The Preparation of Representative Conductive Hydrogels In this example, the preparation of representative conductive hydrogels of the invention is described. Methods for characterizing the hydrogels are also described.

To enable greater control over conductive hydrogel fabrication parameters, such as gel microarchitecture, the present invention provides a one-step synthesis method that incorporates all hydrogel and conductive precursors and initiators in a layered selection of solvents into a single reaction vessel. Useful polymer systems include methacrylate-based monomer systems with poly(HEMA-co-MAA), referred to as H50C, and a methacrylate-based macromer system with PVA-MA-Tau as the gel component, referred to as PVA. In these systems, PEDOT serves as the primary the conductive component. The gel systems containing PEDOT are referred to as H50C-2.5E and PVA-2.5E while their respective control gels are referred to as H50C and PVA.

The monomer systems were mixed in a single vessel to a total volume of 2 mL and comprised of approximately 50% HEMA (v/v) as the hydrogel component, 2.1% MAA (v/v; 16.4:1 HEMA:MAA molar ratio) as immobilized anionic dopant molecules, 2.5% TEGDMA (v/v; 53:1 monomer:crosslinker molar ratio) as crosslinker, 0.25% Irgacure 651 (w/v; 446:1 monomer:initiator molar ratio) as photoinitiator, 2.5% EDOT (v/v; 1:1 EDOT:dopant molar ratio) as an electronic and ionic conductor, 5.7% APS (w/v; 1:1 EDOT:initiator molar ratio) as oxidative initiator/dopant, and 21.3% PBS (pH 7.4) (v/v) and 21.3% alcohol (v/v) for the H50C system as solvents.

The macromer system was mixed in a single vessel to a total volume of 2 mL and comprised of approximately 8% PVA-MA-Tau (w/v) which equates to 0.75% MA (w/v; 36:1 VA repeat:crosslinker molar ratio) as crosslinker and 1.25% taurine (w/v; 18:1 VA repeat:dopant molar ratio) as dopant, 0.1% Irgacure 2959 (w/v; 11:1 crosslinker:initiator molar ratio) as photoinitiator, 2.5% EDOT (v/v; 2.5:1 EDOT:dopant molar ratio), 5.7% APS (w/v; 1:1 EDOT:APS molar ratio) as oxidative initiator/dopant, and 41.3% PBS (Ph 7.4)(v/v) and 41.3% ethanol (v/v) as solvents. Control groups for each system were made by modifying the pre-gel solutions to exclude the EDOT and APS components, replace the methanol/ethanol with DI water, and bring the total volume back to 2 mL.

H50C, H50C-2.5E, PVA, and PVA-2.5E were made by filling a known volume, 70-80 μL, of pre-gel solution into 10 mm diameter silicone molds covered with glass coverslips or into glass capillary tubes and photopolymerized under a UV spot lamp. The monomer system H50C was exposed to 70 mW/cm² UV light for 3 minutes while the macromer system PVA was exposed to 30 mW/cm² UV light for 3 minutes as it was found that these parameters produced stable mechanical and swelling values. The conductive counterparts to these systems, H50C-2.5E and PVA-2.5E, were exposed to the same intensity UV light to their respective controls but for 12 minutes as solid gels did not form up until this duration, suggesting a reduction in methacrylate polymerization efficiency of the one-step system.

During UV exposure, all systems containing EDOT and APS simultaneously experienced gelation of the hydrogel component and polymerization of the EDOT into PEDOT as the gels turned from translucent to light blue to dark blue to black over the course of the 12 minute UV exposure duration. After gelation had occurred, H50C, H50C-2.5E, PVA, and PVA-2.5E gels were removed from their molds and rinsed in DI water for 1 hour to quickly remove unreacted monomers (to limit contamination of measurement devices with potentially hazardous substances). After rinsing, these gels were either analyzed immediately or washed in PBS (pH 7.4) for 1 day or 1 week before analysis to observe changes over time. All gels containing PEDOT:APS, H50C-2.5E and PVA-2.5E, remained in their molds and wrapped in parafilm to limit water loss and then placed in a 4C fridge to allow the PEDOT polymerization to complete. After 1 week, these gels were removed from the glass and prepared for analysis as their respective controls.

Morphological Analysis

To analyze the micro-scale morphological features of each system, gels equilibrated in DI water were cut in half with a sharp razor, frozen at −80° C. and then lyophilized until completely dry. Dehydrated gels were then coated with a thin layer of metal (Au/Pd) and imaged using a scanning electron microscope (SEM) at various magnifications.

Hydration Analysis

The effect of PEDOT incorporation on bulk material hydrogel hydration properties was assessed with block (i.e., non-porous) gel discs. Hydration analysis was carried out by measuring the weights of gels swollen in solvent and after lyophilization (freeze drying) to calculate the swelling ratio via the following equation:

Swelling (%)=(Wₛ−W_{d})/W_{d}×100 (1)

Here Ws refers to the weight of the gel in the solvent swollen state while Wd refers to the weight of the gel in the dehydrated state to calculate swelling ratio as a percent of the solvent component absorbed into the gel with respect to the total mass of the polymer component. Swelling values equal to 100% indicate that the swollen gel contains the same mass of solvent as that of the polymer component in the swollen state.

Mechanical Compression

The effect of PEDOT incorporation on bulk material hydrogel mechanical properties was also assessed with block gel discs. The compression modulus of gels was measured via Instron compression testing with a compression rate of 0.1 mm/min to emulate previous studies investigating the compression modulus of brain tissue. Compression modulus was obtained by calculating the slope of the linear region of the stress vs. strain curve around 10-15% compressive strain.

Electrochemical Characterization

Electrochemical characterization of materials was carried out with a potentiostat connected to electrodes in a 3-electrode cell containing PBS (pH 7.4) as the supporting electrolyte, a platinum mesh or wire as counter electrode, and a silver/silver-chloride (Ag/AgCl) reference electrode. Rod-shaped gel blocks 800 μm in diameter were cut into 3 cm long segments and equilibrated in PBS (pH 7.4) before being insulated with a 200 μm thick layer of shrink-wrapped polyolefin such that one end was flush with the end of the gel and the other extended −1 mm past the insulation for connection to the potentiostat. Insulated gel rods were gently connected to the potentiostat via their exposed end and served as the working electrode in the electrochemical cell. A 2 mm diameter gold electrode or 1 mm diameter platinum electrode served as working electrode controls.

Electrical impedance spectroscopy was carried out across 5-7 orders of magnitude with 5-10 points per decade and a sinusoidal potential at 50 mV root-mean squared. Impedance at 1 kHz was compared across gels for statistical analysis as this is a relevant frequency at which neuronal signals operate. Cyclic voltammetry was carried out with a 50 mV/s scan rate and cathodal charge storage capacity (CSCC) calculated by taking the time integral of the cathodal (negative) current from a single scan cycle and then normalizing to electrode surface area.

Statistics

For comparison of conductive hydrogels, paired T-tests were used to determine if statistically significant differences existed between samples of the same type before and after PEDOT inclusion (e.g. H50C vs H50C-2.5E) with a=0.05. Comparisons with p-values less than 0.05 were designated statistically significant and marked with an asterisk (*). One-way ANOVA was used to compare across sample types (e.g. H50C vs. H50C-2.5E vs. PVA vs. PVA-2.5E) followed by repeated T-tests with a Bonferroni correction to account for multiple comparisons. Comparisons with p-values less than this corrected value were designated statistically significant and marked with a hash (#) or double hash (##) to designate specific comparisons. All experiments were run in triplicate with at least 3 gels per experimental repeat and data are reported as mean±standard deviation.

### Example 2

### Representative Conductive Hydrogels

In this example, representative conductive hydrogels of the invention are identified. The representative conductive hydrogels were prepared as described in Example 1.

Example 1 provides a description of the preparation of conductive hydrogels designated as H50C-2.5E and PVA-2.5E and their corresponding control hydrogels designated as H50C and PVA, respectively.

Other representative conductive hydrogels of the invention and their corresponding control hydrogels include hydrogels with the following designations: HG50SC-2.5E, HG50SC-5E, HG50SC-7.5E, HG25SC-2.5E, HG25SC-3E, and HG25SC-5E. Control hydrogels include HG50S, HG25S, HG50C, and HG25C.

The conductive hydrogels of the invention (e.g., H50C-2.5E) are identified herein by their polymeric composition (e.g., H50C) and the pre-gel concentration of the conductive monomer (e.g., 2.5E, which refers to 2.5% by volume EDOT based on the total volume of the pre-gel composition).

In the above designations the first letters correspond to the hydrogel composition with H=HEMA and G=GMA. Samples designated with an H were 100% HEMA for the hydrogel component while samples designated with HG were a 4:1 mixture of HEMA:GMA. PVA refers to the PVA-MA-Tau macromer.

The numeral after these first letters represent the volume % of the hydrogel component for the monomeric systems (i.e., 50% and 25%). The PVA gels were composed of 8 weight % PVA (with an assumed density of 1.0 g/mL for subsequent volumetric calculations).

The letter following the numeral indicates the type of anionic dopant molecule immobilized throughout the hydrogel network via inclusion of anionic monomeric species. C refers to a carboxylic acid moiety from an AA monomer while S refers to sulfonic acid from an AMPS monomer. SC together indicates both carboxylic acid and sulfonic acid moieties were included in the pre-gel solution. The anionic monomers were always included at 0.1 mol/L, so, for example, HG25S-2.5E had 0.1 mol/L of AMPS included in the pre-gel solution, while HG25SC-2.5E had 0.1 mol/L of AMPS and 0.1 mol/L of AA for a total of 0.2 mol/L anionic dopant in the pre-gel solution.

The final text after the hyphen indicates the volume % of EDOT included into the pre-gel solution at 0% (when “-XE” is absent), 2.5%, 3.0, 5.0%, or 7.5%. E refers to EDOT.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for making a conductive hydrogel, comprising photochemically polymerizing one or more polymerizable hydrogel-forming agents in the presence of a polymerizable conductive monomer to provide a conductive hydrogel.

2. The method of claim 1, wherein the polymerizable hydrogel-forming agents comprise hydrogel-forming monomers and a crosslinking agent.

3. The method of claim 2, wherein the polymerizable hydrogel-forming monomers comprise 2-hydroxyethyl methacrylate (HEMA), methacrylic acid (MAA), and styrene sulfonate (SS), and the crosslinking agent is tetraethyleneglycol dimethacrylate (TEGDMA) or polyethylene glycol diacrylate (PEGDA).

4. The method of claim 1, wherein the polymerizable hydrogel-forming agents comprise hydrogel-forming macromers.

5. The method of claim 4, wherein the hydrogel-forming macromers comprise PVA-MA-Taurine.

6. The method of claim 1, wherein the polymerizable conductive monomer is a polymerizable thiophene.

7. The method of claim 6, wherein the polymerizable thiophene is 3,4-ethylene dioxythiophene (EDOT).

8. A method for making a conductive hydrogel, comprising irradiating a solution comprising:
(a) a polymerizable hydrogel-forming monomer or a polymerizable hydrogel-forming macromer;
(b) a crosslinking agent effective to react with the monomer or macromer to provide a crosslinked hydrogel;
(c) a photoinitiator effective to initiate photopolymerization of the monomer or macromer and the crosslinking agent;
(d) a polymerizable conductive monomer;
(e) an oxidative initiator effective to initiate polymerization of the polymerizable conductive monomer; and
(f) a solvent effective to solubilize (a)-(e),
wherein one or more of the polymerizable hydrogel-forming monomer, polymerizable hydrogel-forming macromer, the crosslinking agent, the photoinitiator, or the oxidative initiator include an anionic or cationic moiety,
wherein the solution is irradiated with light having a wavelength effective to initiate polymerization of the polymerizable hydrogel-forming monomer or macromer and polymerization of the polymerizable conductive monomers, and
wherein the solution is irradiated at a temperature and for a time sufficient to provide a conductive hydrogel.

9. The method of claim 8, wherein the polymerizable hydrogel-forming monomer or polymerizable hydrogel-forming macromer includes an anionic or cationic moiety.

10. The method of claim 8, wherein the oxidative initiator includes an anionic or cationic moiety.

11. The method of claim 8, wherein the polymerizable hydrogel-forming monomer or polymerizable hydrogel-forming macromer and the oxidative initiator include an anionic or cationic moiety.

12. The method of claim 8, wherein the photoinitiator is 2,2-dimethoxy-1,2-diphenylethanone (IRGACURE 651) or 2-hydroxy-4′-(2-hydroxyethoxy)-2-methylpropiophenone (IRGACURE 2959).

13. The method of claim 8, wherein the oxidative initiator is a persulfate (e.g., ammonium persulfate, APS; sodium persulfate, NaPS; potassium persulfate, KPS).

14. The method of claim 8, wherein the solvent is an alcohol.

15. The methods of claim 1 further comprising the use of sphere-templated scaffold fabrication to produce the conductive hydrogel.

16. A liquid resin, comprising:
(a) a polymerizable hydrogel-forming monomer or a polymerizable hydrogel-forming macromer;
(b) a crosslinking agent effective to react with the monomer or macromer to provide a crosslinked hydrogel;
(c) a photoinitiator effective to initiate photopolymerization of the monomer or macromer and the crosslinking agent;
(d) a polymerizable conductive monomer;
(e) an oxidative initiator effective to initiate polymerization of the polymerizable conductive monomer; and
(f) a solvent effective to solubilize (a)-(e),
wherein one or more of the polymerizable hydrogel-forming monomer, polymerizable hydrogel-forming macromer, the crosslinking agent, the photoinitiator, or the oxidative initiator include an anionic or cationic moiety.

17. The method of claim 16, wherein the polymerizable hydrogel-forming monomer or polymerizable hydrogel-forming macromer includes an anionic or cationic moiety.

18. The method of claim 16, wherein the oxidative initiator includes an anionic or cationic moiety.

19. The method of claim 16, wherein the polymerizable hydrogel-forming monomer or polymerizable hydrogel-forming macromer and the oxidative initiator include an anionic or cationic moiety.

20. A method for making a conductive object or conductive pattern, comprising:
(a) 3D printing, micropatterning, or photolithographically or stereolithography patterning a liquid resin of claim 16 to provide a printed or patterned resin; and
(b) irradiating the printed or patterned resin to provide a conductive object or conductive pattern.
